(19) United States
(12) Patent Application Publication
(10) Pub. No.: US 2007/0189978 A1
(43) Pub. Date: Aug. 16, 2007

(54) Compositions and methods for dermally treating musculoskeletal pain

(76) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,133

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/750,683, filed on Dec. 14, 2005.
(60) Provisional application No. 60/577,536, filed on Jun. 7, 2004.

(51) Int. Cl. A61K 9/12; A61K 31/24; A61K 31/192; A61K 31/195

(52) U.S. Cl. 424/45; 514/563; 514/569; 514/570; 514/536

(57) ABSTRACT

The present invention is drawn to solidifying formulations for dermal delivery of a drug for treating musculoskeletal pain, inflammation, joint pain, etc. The formulation can include a drug selected from certain drug classes, a solvent vehicle, and a solidifying agent. The solvent vehicle can include a volatile solvent system having one or more volatile solvent, and a non-volatile solvent system having one or more non-volatile solvent, wherein the evaporation of at least some of the volatile solvent converts the formulation on the skin into a solidified layer and the non-volatile solvent system is capable of facilitating the topical delivery of the drug(s) at therapeutically effective rates over a sustained period of time.

This application claims the benefit of U.S. Provisional Application Nos. 60/750,637 and 60/750,683, each of which was filed on Dec. 14, 2005, and is a continuation-in-part of U.S. Application No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to formulations and methods for treating musculoskeletal pain or inflammation. More particularly, the present invention relates to adhesive formulations having a viscosity suitable for application to a skin surface and which form a transdermal drug-delivering solidified layer on the skin.

### BACKGROUND OF THE INVENTION

It is believed that topically absorbed non-steroidal anti-inflammatory drugs (NSAIDs), local anesthetics, and certain steroids can reduce musculoskeletal pain or inflammation. However, current topical dosage forms for those drugs are not typically adequate for this application. For example, semisolid NSAID and local anesthetic formulations, such as creams and gels, usually contain solvent(s), such as water and ethanol, which are volatile and thus evaporate shortly after application. The evaporation of such solvents can cause significant decrease or even termination of topical drug absorption. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing.

With respect to drug-in-adhesive patches, in order to be delivered appropriately, a drug should have sufficient solubility in the adhesive, as primarily only dissolved drug contributes to the driving force required for skin permeation. Unfortunately, many drugs have low solubility in adhesives that is not high enough to generate sufficient skin permeation driving force over a period of time. In addition, many ingredients, e.g., liquid solvents and permeation enhancers, which could be used to help dissolve the drug or increase the skin permeability, cannot be incorporated into many adhesive matrix systems in sufficient quantities to be effective, as many of these materials may adversely alter the adhesive properties of the matrix. As such, the selection and allowable quantities of additives, enhancers, excipients, or the like in adhesive-based matrix patches can be limited. To illustrate, for many drugs, optimal transdermal flux can be achieved when the drug is dissolved in certain liquid solvent systems, but a thin layer of adhesive in a typical matrix patch often cannot hold enough appropriate drug and/or additives to be therapeutically effective. Further, the properties of the adhesives, such as adherence, coherence, and tackiness, can also be significantly changed by the presence of liquid solvents.

With regard to liquid reservoir patches, even when a drug is compatible with a particular liquid or semisolid solvent system carried by the thin bag of the patch, the solvent system still has to be compatible to the adhesive layer coated on the permeable or the semi-permeable membrane otherwise the drug may be adversely affected by the adhesive layer or the drug/solvent system may reduce the tackiness of the adhesive layer. In addition to these dosage form considerations, reservoir patches are usually more expensive to manufacture than matrix patches.

Another shortcoming of dermal (including transdermal) patches is that they are usually not stretchable or flexible, as the backing film (in matrix patches) and the thin fluid bag (in reservoir patches) are typically made of polyethylene or polyester, both of which are relatively non-stretchable materials. If the patch is applied on a skin area that is significantly stretched during body movements, such as a joint, separation between the patch and skin may occur, thereby compromising the delivery of the drug. In addition, a patch present on a skin surface may hinder the expansion of the skin during body movements and cause discomfort. For these additional reasons, patches are not ideal dosage forms for skin areas over muscle and joints that are subject to expansion and stretch during body movements.

In view of the shortcomings of the current delivery systems, it would be desirable to provide systems and/or methods that i) can provide more sustained delivery of NSAIDs, local anesthetics, or certain steroids over long periods of time; ii) are not vulnerable to unintentional removal by contact with clothing, other objects, or people for the duration of the application time; iii) can be applied to a skin area subject to stretch and expansion without causing discomfort or poor contact to skin; and/or iv) can be conveniently removed after application and use.

### SUMMARY OF THE INVENTION

It has been recognized that it would be advantageous to treat musculoskeletal pain and/or inflammation by providing topical delivery of drugs from certain classes, e.g., NSAID, local anesthetic, or steroid formulations, in the form of adhesive solidifying formulations having a viscosity suitable for application to a skin surface as a layer and which form a drug-delivering solidified adhesive layer on the skin. In one embodiment, a formulation for treating musculoskeletal pain or inflammation can comprise a drug suitable for treating musculoskeletal pain or inflammation, a solvent vehicle, and a solidifying agent. The solvent vehicle can comprise a volatile solvent system comprising at least one volatile solvent, and a non-volatile solvent system comprising at least one non-volatile solvent, wherein the non-volatile solvent system is capable of facilitating transdermal delivery of the drug at a therapeutically effective rate over a sustained period of time. The formulation can have a viscosity suitable for application and adhesion to a skin surface prior to evaporation of the volatile solvent system, and further, the formulation applied to the skin surface can form a solidified layer after at least partial evaporation of the volatile solvent system. The drug can continue to be delivered at the therapeutically effective rate to treat musculoskeletal pain or inflammation after the volatile solvent system is at least substantially evaporated.

In another embodiment, a method of dermally delivering a drug for treating pain or inflammation of joints or muscles can comprise applying a formulation to a skin surface. The formulation can comprise a drug suitable for treating musculoskeletal pain or inflammation, a solvent vehicle, and a solidifying agent. The solvent vehicle can comprise a volatile solvent system comprising at least one volatile solvent, and a non-volatile solvent system comprising at least one non-volatile solvent, wherein the non-volatile solvent system is capable of facilitating dermal delivery of the drug at a therapeutically effective rate over a sustained period of time. The formulation can have a viscosity suitable for application and adhesion to the skin surface prior to evaporation of the volatile solvent system. Additional steps include solidifying the formulation to form a solidified layer on the skin surface by at least partial evaporation of the volatile solvent system; and dermally delivering the drug from the solidified layer to the skin surface at therapeutically effective rates for treating the pain or inflammation of joints or muscles over a sustained period of time.

In another embodiment, a solidified layer for treating musculoskeletal pain or inflammation can comprise a drug effective for treating musculoskeletal pain or inflammation, a non-volatile solvent system, and a solidifying agent. The non-volatile solvent system can include at least one non-volatile solvent, wherein the non-volatile solvent system is capable of facilitating the delivery of the drug at therapeutically effective rates over a sustained period of time. Additionally, the solidified layer preferably can be stretchable by 5% in at least one direction without cracking, breaking, and/or separating from a skin surface to which the layer is applied.

In another embodiment, a formulation for treating musculoskeletal pain or inflammation can comprise ropivacaine, a solvent vehicle, and a solidifying agent. The solvent vehicle can include a volatile solvent system including at least one volatile solvent, and a non-volatile solvent system including at least one solvent selected from the group consisting of triacetin, span 20, isostearic acid, and combinations thereof. The ropivacaine can either be in base or salt form. The formulation has a viscosity suitable for application to a skin surface prior to evaporation of the volatile solvent system, and can be applied to the skin surface to form a solidified, coherent, flexible, and continuous layer after at least partial evaporation of the volatile solvent system. Further, the ropivacaine can continue to be delivered at a transdermal flux of at least 5 mcg/cm²/hour after the volatile solvent system is at least substantially all evaporated. In another embodiment, the transdermal flux can be at least 10 mcg/cm²/hour after the volatile solvent system is at least substantially all evaporated from the solidified layer.

In another embodiment, a formulation for treating musculoskeletal pain or inflammation can comprise lidocaine, a solvent vehicle, and a solidifying agent. The solvent vehicle can include a volatile solvent system including at least one volatile solvent, and a non-volatile solvent system including at least one solvent selected from the group consisting of propylene glycol and dipropylene glycol. The lidocaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The lidocaine can continue to be delivered at a transdermal flux of at least 20 mcg/cm²/hour after the volatile solvent system is at least substantially all evaporated fro the solidified layer.

In another embodiment, a formulation for treating musculoskeletal pain or inflammation can comprise ketoprofen, a solvent vehicle, and a solidifying agent. The solidifying agent can comprise a volatile solvent system including at least one volatile solvent, and a non-volatile solvent system including at least one solvent selected from the group consisting of propylene glycol and glycerol, isostearic acid, and triacetin. The ketoprofen can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The ketoprofen can continue to be delivered at a transdermal flux of at least 10 mcg/cm²/hour after the volatile solvent system is at least substantially all evaporated fro the solidified layer.

In still another embodiment, a formulation for treating musculoskeletal pain or inflammation can comprise tetracaine, a solvent vehicle, and a solidifying agent. The solvent vehicle can comprise a volatile solvent system including at least one volatile solvent, and a non-volatile solvent system including at least one solvent selected from the group consisting of propylene glycol and isostearic acid. The tetracaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The tetracaine can continue to be delivered at a transdermal flux of at least 5 mcg/cm²/hour after the volatile solvent system is at least substantially all evaporated fro the solidified layer.

In yet another embodiment, a formulation for treating musculoskeletal pain or inflammation can comprise lidocaine and tetracaine, a solvent vehicle, and a solidifying agent. The solvent vehicle can comprise volatile solvent system including at least one volatile solvent, and a non-volatile solvent system including at least one solvent selected from the group consisting of propylene glycol and dipropylene glycol, and isostearic acid. The tetracaine and lidocaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The tetracaine and lidocaine can continue to be delivered at a transdermal flux of at least 5 mcg/cm²/hour, respectively, after the volatile solvent system is at least substantially all evaporated from the solidified layer.

In another embodiment, a formulation for treating musculoskeletal pain or inflammation, can comprise a drug include at least one member from the group consisting of lidocaine, tetracaine, ropivacaine, ketoprofen, diclofenac, and combinations thereof; a solvent vehicle; and a solidifying agent. The solvent vehicle can comprise a volatile solvent system including a volatile solvent whose boiling point is below 20° C., and a non-volatile solvent system comprising at least one non-volatile solvent. The formulation can have a viscosity suitable for application to a skin surface prior to evaporation of the volatile solvent system, and can be applied to the skin surface to a solidified, coherent, flexible and continuous layer after at least partial evaporation of the volatile solvent system. The drug can continue to be delivered at a therapeutically effective rate after the volatile solvent system is at least substantially all evaporated.

Additional features and advantages of the invention will be apparent from the following detailed description and figures which illustrate, by way of example, features of the invention.

### BRIEF DESCRIPTION OF THE DRAWINGS

FIG. 1 is a graphical representation of the cumulative amount of diclofenac delivered transdermally across human cadaver skin over time from a peel formulation in accordance with embodiments of the present invention where steady-state delivery is shown over 28 hours; and

FIG. 2 is a graphical representation of the cumulative amount of ropivacaine delivered transdermally across human cadaver skin over time from a peel formulation with similar composition in accordance with embodiments of the present invention, where steady-state delivery is shown over 30 hours.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

The term “musculoskeletal pain or inflammation” includes pain and/or inflammation of joints, tendons, ligaments, muscles, bones, synovial fluids, and/or soft tissues which are part of the musculoskeletal system.

The term “drug(s)” refers to active agents that can be used with the formulations of the present invention, including NSAIDs, local anesthetics, steroid drugs, and/or 5-HT2A receptor antagonists and any bioactive agents whose presence in the musculoskeletal tissue, e.g. joints, muscles, bones, synovial fluids, soft tissues, etc., can alleviate pain, inflammation, or discomfort. An example of a 5-HT2A receptor antagonist includes but is not limited to ketanserin. Examples of NSAIDS include but are not limited to ketoprofen, piroxicam, diclofenac, indomethacin, and COX inhibitors. Examples of local anesthetics include but are not limited to lidocaine, bupivacaine, ropivacaine, and tetracaine. Examples of steroid drugs for use in the present invention include but are not limited to dexamethasone, hydrocortisone, prednisone, prednisolone, methylprednisolone, halobetasol propionate, betamethasone dipropionate, betamethasone, prodrugs thereof, or combinations thereof. When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The term “NSAID” or “non-steroidal anti-inflammatory drug” include all the non-steroidal anti-inflammatory agents, general COX inhibitors, COX-2 selective inhibitors, and COX-3 selective inhibitors.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values set forth herein can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic sufficient levels of the drug across, onto or into the subject's skin when the non-volatile solvent system is saturated with the drug. For systemically targeted drugs, a flux enabling non-volatile solvent system is a non-volatile solvent system that can provide therapetucially sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

For lidocaine base, a non-volatile solvent system would be “flux enabling” if it is capable of generating a flux of at least about 20 mcg/cm²/hour in a setup same or similar to that described in Example 1. For tetracaine and ropivacaine bases, a non-volatile solvent system would be “flux enabling” if it is capable of generating a flux of at least about 5 mcg/cm²/hour in a setup the same or similar to that described in Example 1. For ketoprofen and diclofenac, a non-volatile solvent system would be “flux enabling” if it is capable of generating a flux of at least about 5 mcg/cm²/hour in the same or similar setup to that described in Example 1.

For example, the importance of selecting an appropriate non-volatile solvent is demonstrated in Table 1. The flux of ropivacaine (a local anesthetic agent effective in treating neuropathic pain) from saturated glycerol, isostearic acid (ISA) alone and ISA+trolamine, and ISA+trolamine peel are presented in Table 1. Flux values were generated in an in vitro experiment described below in Example 1. The estimated therapeutically beneficial ropicavaine flux is 5-10 mcg/cm²/h.

TABLE 1

| Non-volatile solvent | In vitro flux (mcg/cm²/h)* |
|---|---|
| ISA | 11 ± 2 |
| ISA + 20% Trolamine | 43 ± 7 |
| ISA + Trolamine peel | 32 ± 2 |
| Glycerol | 1.2 ± 0.7 |

Estimated therapeutically beneficial flux = 5-10
mcg/cm²/h
*In vitro flux values represent the mean and st. dev. of three determinations.

In vitro flux results of ropivacaine from ISA, and ISA+trolamine are examples of a suitable non-volatile solvent and glycerol is an example of an unsuitable non-volatile solvent. When incorporated into a peel formulation, the suitable non-volatile solvent dictates the flux-generating power of the formulation. It should be noted that a “non-volatile solvent system suitable for the selected drug” can be a single chemical substance or a mixture of two or more chemical substances. As can be seen above, the non-volatile solvent system of ISA+trolamine can generate more flux than the non-volatile solvent system of pure ISA, though both are probably suitable for certain applications.

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine. “Therapeutically effective flux” is defined as the permeation flux of the selected drug that delivers sufficient amount of drug into or across the skin to be clinically beneficial in that some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400 cm²/10 hour, which equals 1 mcg/cm²/hr. By this definition, different drugs have different “therapeutically effective flux.” Additionally, therapeutically effective flux may be different in different subjects and or at different times for even the same subject. However, for each drug, there is usually a consensus among the skilled in the art on the range of doses or fluxes that are sufficient in most subjects at most times.

The term “plasticizing”, “plasticizing” in relation to non-volatile solvent (or a non-volatile solvent system) and the solidifying agent is defined as a non-volatile solvent (or a non-volatile solvent system) that acts as a plasticizer for the solidifying agent. A “plasticizer” is an agent which is capable of providing the flexibility and/or elasticity of the solidified formulation layer after the volatile solvent system has at least substantially evaporated. Plasticizers also have the capability to reduce the brittleness of solidified formulation by making it more flexible and/or elastic. For example, propylene glycol is a plasticizing non-volatile solvent for a solidifying formulation with ketoprofen as the drug and polyvinyl alcohol as the selected solidifying agent. However, propylene glycol in a solidifying formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table C is a 9:1 for propylene glycol : isostearic acid mixture that generated much higher clobetasol flux than propylene glycol or ISA alone (see Table B). Therefore, the 9:1 propylene glycol:isostearic acid mixture is a “high flux-enabling non-volatile solvent” but propylene glycol or isostearic acid alone is not.

The term “adhesion” or “adhesive” when referring to a solidified layer herein refers to sufficient adhesion between the solidified layer and the skin so that the layer does not fall off the skin during intended use on most subjects. Thus, “adhesive” or the like when used to describe the solidified layer means the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the current invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole. “Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive peelable formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

The term “adhesive” in relation to the solidified layer means it is adhesive to the skin on which the original formulation was applied, and not necessarily, and preferably not, adhesive on the other side to other objects. “Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and various aliphatic resins and aromatic resins.

The terms “washable” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol, or combinations thereof. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above. “Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, in one embodiment, a formulation for treating musculoskeletal pain or inflammation can comprise a drug suitable for treating musculoskeletal pain or inflammation, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating transdermal delivery of the drug at a therapeutically effective rate over a sustained period of time. The formulation can have a viscosity suitable for application and adhesion to a skin surface as a layer prior to evaporation of the volatile solvent system, and further, the formulation applied to the skin surface can form a solidified layer after at least partial evaporation of the volatile solvent system. The drug can continue to be delivered at the therapeutically effective rate to treat musculoskeletal pain or inflammation after the volatile solvent system is at least substantially evaporated.

In another embodiment, a method of dermally delivering a drug for treating pain or inflammation of joints or muscles can comprise applying an adhesive solidifying formulation to a skin surface adjacent to the tissue suffering from the pain or inflammation (for example, the skin surface of a knee suffering from arthritis or the skin of lower back which is suffering from lower back pain). The adhesive solidifying formulation can comprise a drug suitable for treating musculoskeletal pain or inflammation, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating dermal delivery of the drug at a therapeutically effective rate over a sustained period of time. The formulation can have a viscosity suitable for application and adhesion to the skin surface prior to evaporation of the volatile solvent system. Additional steps include solidifying the formulation to form a solidified layer on the skin surface by at least partial evaporation of the volatile solvent system; and dermally delivering the drug from the solidified layer to the skin surface at therapeutically effective rates for treating the pain or inflammation of joints or muscles over a sustained period of time.

In another embodiment, a solidified layer for treating musculoskeletal pain or inflammation can comprise a drug effective for treating musculoskeletal pain or inflammation, a non-volatile solvent system, and a solidifying agent. The non-volatile solvent system can include at least one non-volatile solvent, wherein the non-volatile solvent system is capable of facilitating the delivery of the drug at therapeutically effective rates over a sustained period of time. Additionally, the solidified layer can be stretchable by 5% in at least one direction without cracking, breaking, and/or separating from a skin surface to which the layer is applied.

In another embodiment, a formulation for treating musculoskeletal pain or inflammation can comprise ropivacaine, a solvent vehicle, and a solidifying agent. The solvent vehicle can include a volatile solvent system including at least one volatile solvent, and a non-volatile solvent system including at least one solvent selected from the group consisting of triacetin, span 20, isostearic acid, or combinations thereof. The ropivacaine can either be in base or salt form. The formulation has a viscosity suitable for application to a skin surface prior to evaporation of the volatile solvent system, and can be applied to the skin surface to form a solidified, coherent, flexible, and continuous layer after at least partial evaporation of the volatile solvent system. Further, the ropivacaine can continue to be delivered at a transdermal flux of at least 5 mcg/cm²/hour after the volatile solvent system is at least substantially all evaporated. In another embodiment, the transdermal flux can be at least 10 mcg/cm²/hour after the volatile solvent system is at least substantially all evaporated from the solidified layer.

In another embodiment, a formulation for treating musculoskeletal pain or inflammation can comprise lidocaine, a solvent vehicle, and a solidifying agent. The solvent vehicle can include a volatile solvent system including at least one volatile solvent, and a non-volatile solvent system including at least one solvent selected from the group consisting of propylene glycol and dipropylene glycol. The lidocaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The lidocaine can continue to be delivered at a transdermal flux of at least 20 mcg/cm²/hour after the volatile solvent system is at least substantially all evaporated fro the solidified layer.

In another embodiment, a formulation for treating musculoskeletal pain or inflammation can comprise ketoprofen, a solvent vehicle, and a solidifying agent. The solidifying agent can comprise a volatile solvent system including at least one volatile solvent, and a non-volatile solvent system including at least one solvent selected from the group consisting of propylene glycol and glycerol, isostearic acid, and triacetin. The ketoprofen can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The ketoprofen can continue to be delivered at a transdermal flux of at least 10 mcg/cm²/hour after the volatile solvent system is at least substantially all evaporated fro the solidified layer.

In still another embodiment, a formulation for treating musculoskeletal pain or inflammation can comprise tetracaine, a solvent vehicle, and a solidifying agent. The solvent vehicle can comprise a volatile solvent system including at least one volatile solvent, and a non-volatile solvent system including at least one solvent selected from the group consisting of propylene glycol and isostearic acid. The tetracaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The tetracaine can continue to be delivered at a transdermal flux of at least 5 mcg/cm²/hour after the volatile solvent system is at least substantially all evaporated fro the solidified layer.

In yet another embodiment, a formulation for treating musculoskeletal pain or inflammation can comprise lidocaine and tetracaine, a solvent vehicle, and a solidifying agent. The solvent vehicle can comprise volatile solvent system including at least one volatile solvent, and a non-volatile solvent system including at least one solvent selected from the group consisting of propylene glycol and dipropylene glycol, and isostearic acid. The tetracaine and lidocaine can be in either base or salt form. The formulation can have a viscosity suitable for application to a skin surface prior to evaporation of the volatile solvent system, and can be applied to the skin surface to form a solidified, coherent, flexible and continuous layer after at least partial evaporation of the volatile solvent system. The tetracaine and lidocaine can continue to be delivered at a transdermal flux of at least 5 mcg/cm²/hour, respectively, after the volatile solvent system is at least substantially all evaporated from the solidified layer.

In another embodiment, a formulation for treating musculoskeletal pain or inflammation, can comprise a drug include at least one member from the group consisting of lidocaine, tetracaine, ropivacaine, ketoprofen, diclofenac, or combinations thereof; a solvent vehicle; and a solidifying agent. The solvent vehicle can comprise a volatile solvent system including a volatile solvent whose boiling point is below 20° C., and a non-volatile solvent system comprising at least one non-volatile solvent. The formulation can have a viscosity suitable for application to a skin surface prior to evaporation of the volatile solvent system, and can be applied to the skin surface to a solidified, coherent, flexible and continuous layer after at least partial evaporation of the volatile solvent system. The drug can continue to be delivered at a therapeutically effective rate after the volatile solvent system is at least substantially all evaporated.

Thus, the present invention is related to novel formulations, methods, and solidified layers that are typically in the initial form of semi-solids (including creams, gels, pastes, ointments, and other viscous liquids), which can be easily applied onto the skin as a layer, and can quickly (from 15 seconds to 4 minutes under standard skin and ambient conditions) to moderately quickly (from 4 to 15 minutes under standard skin and ambient conditions) change into a solidified layer, e.g., a coherent and soft solid layer for drug delivery for reducing musculoskeletal pain. The solidified layer thus formed is capable of delivering drug into or across the skin at therapeutically effective rates, over a sustained period of time, e.g., hours to tens of hours, so that most of the drug delivery occurs after the solidified layer is formed. Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus capable of maintaining good contact with the skin surface, even if the skin is stretched during body movement, such as at a knee, finger, elbow, wrist, finger, hip, neck, back, joints, or other areas where skin is typically stretched.

In selecting or formulating the various components that can be used, e.g., drug, solvent vehicle of volatile solvent system and non-volatile solvent system, solidifying agent(s), etc., certain variables can be considered. For example, the volatile solvent system can be selected from pharmaceutically or cosmetically acceptable solvents known in the art. In one embodiment of the present invention, the volatile solvent system can include ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, or combinations thereof. In another embodiment of the present invention, the volatile solvent system can include iso-amyl acetate, denatured alcohol, methanol, propanol, isobutene, pentane, hexane, chlorobutanol, turpentine, cytopentasiloxane, cyclomethicone, methyl ethyl ketone, or combinations thereof. The volatile solvent system can include a mixture or combination of any of the volatile solvents set forth in the embodiments above.

These volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %.

The non-volatile solvent system can also be chosen or formulated to be compatible with the solidifying agent, the drug, the volatile solvent, and any other ingredients that may be present. For example, the solidifying agent can be chosen so that it is dispersible or soluble in the non-volatile solvent system. Most non-volatile solvent systems and solvent vehicles as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a formulation containing PVA. With this combination, Span 20 can separate out of the formulation and form an oily layer on the surface of the solidified layer. Thus, appropriate solidifying agent/non-volatile solvent selections are desirable in developing a viable formulation and compatible combinations.

Non-volatile solvent(s) that can be used alone or in combination to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In another embodiment the non-volatile solvent system can include benzoic acid, butyl alcohol, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers such as PEG-cetyl ether, PEG-stearyl ether, PEG-sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment the non-volatile solvent system can include a combination or mixture of non-volatile solvents set forth in the any of the above discussed embodiments.

In addition to these and other considerations, the non-volatile solvent system can also serve as plasticizer in the adhesive formulation so that when the solidified layer is formed, the layer is flexible, stretchable, and/or otherwise skin friendly. Plasticizers also have the capability to reduce the brittleness of solidified formulation by making it more flexible and/or elastic. For example, propylene glycol is a plasticizing non-volatile solvent for a solidified layer with polyvinyl alcohol as the selected solidifying agent and ketoprofen as the drug. However, propylene glycol in a solidifying formulation with Gantrez S-97 or Avalure UR 405 as solidifying agents does not provide the same plasticizing effect. Therefore, whether a given non-volatile solvent is “plasticizing” depends on which solidifying agent(s) is selected.

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the volatile solvent is capable of irritating the skin, it would be helpful to use a non-volatile solvent that is capable of reducing skin irritation. Examples of solvents that are known to be capable of preventing or reducing skin irritation include, but are not limited to, glycerin, honey, and propylene glycol.

The formulations of the present invention may also contain two or more non-volatile solvents that independently are not adequate non-volatile solvents for a drug but when formulated together become an adequate non-volatile solvent. One possible reason for these initially non adequate non-volatile solvents to become adequate non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and propylene glycol/isostearic acid.

The selection of the solidifying agent can also be carried out in consideration of the other components present in the adhesive formulation. An appropriate solidifying agent is compatible with the formulation such that the formulation is in liquid or semi-liquid state (e.g. cream, paste, gel, ointment) before any evaporation of the volatile solvent(s) and becomes a soft, coherent adhesive solidified layer after the evaporation of at least some of the volatile solvent(s). The solidifying agent can be selected or formulated to be compatible with the drug and the solvent vehicle (including the volatile solvent(s) and the non-volatile solvent system), as well as provide desired physical properties to the solidified layer once it is formed. Depending on the drug, solvent vehicle, and/or other components that may be present, the solidifying agent can be selected from a variety of agents. In one embodiment, the solidifying agent can include polyvinyl alcohol with a MW range of 20,000-70,000 (Amresco), esters of polyvinylmethylether/maleic anhydride copolymer (ISP Gantrez ES-425 and Gantrez ES-225) with a MW range of 80,000-160,000, neutral copolymer of butyl methacrylate and methyl methacrylate (Degussa Plastoid B) with a MW range of 120,000-180,000, dimethylaminoethyl methacrylate-butyl methacrylate-methyl methacrylate copolymer (Degussa Eudragit E100) with a MW range of 100,000-200,000, ethyl acrylate-methyl methacrylate-trimethylammonioethyl methacrylate chloride copolymer with a MW greater than 5,000 or similar MW to Eudragit RLPO (Degussa), Zein (prolamine) with a MW greater than 5,000 such as Zein with a MW around 35,000 (Freeman industries), pregelatinized starch having a MW similar to Instant Pure-Cote B793 (Grain Processing Corporation), ethyl cellulose MW greater than 5,000 or MW similar to Aqualon EC N7, N10, N14, N22, N50, or N100 (Hercules), fish gelatin having a MW 20,000-250,000 (Norland Products), gelatin, other animal sources with MW greater than 5,000, acrylates/octylacrylamide copolymer MW greater than 5,000 or MW similar to National Starch, and Chemical Dermacryl 79.

In another embodiment the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, or combinations thereof. In a further embodiment the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid), or combinations thereof. In another embodiment, the solidifying agent can include a combination of solidifying agents set forth in the any of the above discussed embodiments. Other polymers may also be suitable as the solidifying agent, depending on the solvent vehicle components, the drug, and the specific functional requirements of the given formulation. Other polymers may also be suitable as the solidifying agent, depending on the solvent vehicle components, the drug, and the specific functional requirements of the given formulation.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, Copolymer of Acrylsan alkyl/Octylacrylamido (Dermacryl 79), and various aliphatic resins and aromatic resins.

The non-volatile solvent system and the solidifying agent are preferably compatible with each other. Compatibility can be defined as i) the solidifying agent does not substantially negatively influence the function of the non-volatile solvent system; ii) the solidifying agent can hold the non-volatile solvent system in the solidified layer so that substantially no non-volatile solvent oozes out of the layer, and iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent can be from about 0.1:1 to about 10:1, or from about 0.5:1 to about 2:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface of the solidified layer. If the drug is very potent and the solidified layer has very high tensile strength, a layer as thin as 0.01 mm may be sufficient. If the drug has rather low potency and the solidified layer has low tensile strength, a layer as thick as 2-3 mm may be needed. Thus, for most drugs and formulations, the appropriate thickness can be from about 0.01 mm to about 3 mm, but more typically, from about 0.05 mm to about 1 mm.

In some embodiments, the flexibility and stretchability of a solidified layer, or optionally solidified peelable layer, can be desirable. Skin areas over joints and certain muscle groups are often significantly stretched during body movements. Such movement prevents non-stretchable patches from maintaining good skin contact. Lotions, ointments, creams, gels, pastes, or the like also may not be suitable for use for the reasons cited above. As such, in transdermal delivery of NSAIDs and other drugs for treating musculoskeletal pain in joints and/or muscles, the solidifying formulations of the present invention can offer unique advantages and benefits.

A further feature of the solid-forming formulations is related to the drying time. If a formulation dries too quickly, the user may not have sufficient time to spread the formulation into a thin layer on the skin surface before the formulation is solidified, leading to poor skin contact. If the formulation dries too slowly, the subject may have to wait a long time before resuming normal activities (e.g. putting clothing on) that may remove un-solidified formulation. Thus, it is desirable for the drying time to be longer than about 15 seconds but shorter than about 15 minutes, and preferably from about 0.5 minutes to about 4 minutes.

Another feature of the formulations of the current invention is related to solidifying formulations comprising a drug for musculoskeletal pain or inflammation of joint or muscles, a non-volatile solvent system comprising at least one non-volatile solvent, a solidifying agent, and a volatile solvent system comprising a volatile solvent whose boiling point is below 20 C (such a solvent is referred to as gaseous volatile solvent). The formulation can be stored in a pressurized container and be sprayed on the skin surface with the help of the gaseous volatile solvent. Some hydrofluorocarbons commonly used as gaseous volatile solvents in pharmaceutical or cosmetic industries can work in this design. More specifically, the gaseous volatile solvents may include, but not limited to dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof. The formulation may also be expelled out of the container and applied on the skin via a manual pump. Formulations including a gaseous volatile solvent are expected to “dry” much faster. Spraying the formulation onto the skin suffering from musculoskeletal pain or inflammation of joints or muscles can avoid touching the skin with an applicator which can cause discomfort to hypersensitive skin and provide an easier means of application of the formulation to a body surface which is inconvenient to reach with an applicator.

The formulations of the current invention may further comprise a pH modifying agent for adjusting the pH of the formulation to a point or a range most suitable for the delivery of the drug. This feature can be important for a drug that is ionizable.

The adhesion to skin and elasticity of the material is such that the solidified layer may not easily separate from the skin. For example, in one embodiment, the solidified layer can be stretched in at least one direction by up to about 5% or even 10% or more without cracking, breaking, or separating form a skin surface to which the solidified layer is applied.

These and other advantage can be summarized by the following non-limiting application embodiments. The solidified formulation layer of the present invention can be prepared in an initial form that is easy to apply as a semisolid dosage form. Additionally, the dosage form can be applied to be relatively thick and can contain much more active drug than a typical layer of traditional cream, gel, lotion, ointment, paste, etc., and further, is not as subject to unintentional removal. After the evaporation of the volatile solvent(s) and the formation of the solidified layer, the drug in the solidified layer can be delivered at desired delivery rates over sustained periods of time. Further, as the solidified layer remains adhesive and can be peelable, easy removal of the solidified layer can occur, usually without the aid of a solvent or surfactant. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched by 5% or even 10% or greater in one direction without cracking, breaking, and/or separating form a skin surface to which the solidified layer is applied. Specific examples of applications that can benefit from the systems, formulations, and methods of the present invention are as follows. In one embodiment, a solidified layer including ketoprofen, diclofanec, or another NSAID, or lidocaine, ropivacaine, or another local anesthetic, can be formulated for treating acute injuries of joints such as joints of the angle, knee, wrist, back, hip, and fingers. In another embodiment, a solidified layer with the same active drugs can be used to treat chronic disorders, such as arthritis (including osteoarthritis and rheumatoid arthritis) induced pain of the finger and/or toe joints.

Still another embodiment involves a peel formulation containing a drug selected from the NSAID class, such as ketoprofen, piroxicam, diclofenac, and indomethacin, which is applied topically to treat symptoms of back pain, muscle tension, or myofascial pain or a combination thereof. The NSAID is gradually released from the formulation to provide pain relief over a sustained period of time. The formulation can become a coherent, soft solid after about 5 minutes and remains adhered to the body surface for the length of its application. It is easily removed any time after drying without leaving residual formulation on the skin surface.

In another embodiment, solidifying formulations for the delivery of drugs that treat the causes or symptoms of diseases involving joints and muscles can also benefit from the systems, formulations, and methods of the present invention. Such diseases that may be applicable include, but not limited to, osteoarthritis (OA), rheumatoid arthritis (RA), joint and skeletal pain of various other causes, myofascial pain, muscular pain, and sports injuries. Drugs or drug classes that can be used for such applications include, but are not limited to, non-steroidal anti-inflammatory drugs (NSAIDs) such as ketoprofen, piroxicam, diclofenac, and indomethacin; COX inhibitors such as non-selective COX inhibitors, COX-2 selective NSAIDs and agents, COX-3 selective NSAIDs and agents; local anesthetics such as lidocaine, bupivacaine, ropivacaine, and tetracaine; 5HT-2A receptor antagonists such as ketanserin; and steroids such as dexamethasone, hydrocortisone, prednisone, prednisolone, methylprednisolone, halobetasol propionate, betamethasone dipropionate, betamethasone, prodrugs thereof, or combinations thereof.

The solidifying formulations and the methods of the current invention are expected to be particularly useful for treating inflammation and/or pain of small joints such as the joints of toes, wrists, ankles, elbow, and especially fingers, as well as chronic musculoskeletal pain that is not necessarily associated with inflammation. Because the pathway from the skin surface to the joints are shorter for smaller joints, therapeutically beneficial amounts of the drugs are more likely reach smaller joints before being taken away by the blood circulation. In addition, as the fingers are often used, bent, and contacted by many objects during normal activities, it is difficult to keep a conventional dosage form or formulation, such as a patch or cream, on the fingers. Furthermore, some physical therapy devices, such as ThermaCare™ heating pads, are too big for finger joints. Therefore, there are many unmet needs for treating the pain or inflammation of finger joints. By applying a drug formulation to the skin overlying affected joints or muscles, the drug can penetrate the skin and directly enter the target tissues (before being taken away by the blood circulation) and establish therapeutic local tissue concentrations without causing significantly high systemic drug concentrations that are associated with adverse side effects. Under such a scenario, it would be easier to deliver the drugs into the tissues of the smaller joints, including the joints of the wrist, elbow, ankle, toe, and particularly the finger, than to that of larger joints such as knees and hips, due to the short pathway between the skin surface and the small joints. Therefore, one method of the present invention uses the solidifying formulations containing NSAID(s), local anesthetic(s), and/or steroid(s) for treating inflammation or pain of small joints, and particularly of finger joints. This being stated, treatment of larger joints or areas of the body can also be treated, such as the back, neck, shoulder, or hip, is also efficacious.

As a further note, it is a unique feature of the solidified layers of the present invention that they can keep a substantial amount of the non-volatile solvent system, which is optimized for delivering the drug, on the body surface. This feature can provide unique advantages over existing products. For example, in some semi-solid formulations, upon application to a skin surface the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membranes as noted for the in vitro flux studies described in herein. Hairless mouse skin (HMS) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Example 2

Formulations of ropivacaine (base) in various non-volatile solvent systems are evaluated. Excess ropivacaine is present. The permeation of ropivacaine from the test formulations through HMS is presented in Table 2 below.

TABLE 2

| | Skin Flux* |
|---|---|
| Non-volatile solvent system | (mcg/cm²/h) |
| Glycerol | 1.2 ± 0.7 |
| Tween 20 | 2.4 ± 0.1 |
| Mineral Oil | 8.9 ± 0.6 |
| ISA (Isostearic Acid) | 11 ± 2 |
| Span 20 | 26 ± 8 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of ropivacaine base from the above non-volatile solvents are obtained by placing 200 mcL on the stratum corneum side (donor) of hairless mouse skin. The in vitro studies are carried out as described in Example 1. From Table 2, the non-volatile solvents glycerol, and Tween 20 had low steady state flux values and would not be considered “flux-enabling”. However, mineral oil and isostearic acid are flux-enabling solvents and are good candidates for evaluation with solidifying agents and volatile solvents to design an acceptable peel formulation. Surprisingly Span 20 has much higher steady state flux values and would also qualify as a high flux-enabling solvent.

### Example 3

Formulations of diclofenac sodium in various non-volatile solvent systems are evaluated. Excess diclofenac sodium is present. The permeation of diclodenac sodium from the test formulations through HMS is presented in Table 3 below.

TABLE 3

| | Skin Flux* |
|---|---|
| Non-volatile solvent system | (mcg/cm²/h) |
| Glycerol | 1.7 ± 0.3 |
| Isopropyl Myristate | 13 ± 3 |
| Ethyl Oleate | 14 ± 4 |
| Propylene Glycol | 30 ± 30 |
| Span 20 | 98 ± 20 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of diclofenac sodium from the above non-volatile solvents are obtained by placing 200 mcL on the stratum corneum side (donor) of hairless mouse skin. The in vitro studies are carried out as described in Example 1. From Table 3, the non-volatile solvent glycerol has a steady state flux value comparable to the estimated therapeutic steady state flux value of 1 mcg/cm²/h and may be considered a flux-enabling solvent. However, the steady state flux values of isopropyl myristate, ethyl oleate, propylene glycol, and Span 20 are at least 10 times the flux value reported for glycerol and are considered flux enabling.

### Example 4

Formulations of diclofenac acid in various non-volatile solvent systems are evaluated. Excess diclofenac acid is present. The permeation of diclofenac from the test formulations through HMS is presented in Table 4 below.

TABLE 4

| | Skin Flux* |
|---|---|
| Non-volatile solvent system | (mcg/cm²/h) |
| Glycerol | 0 |
| Isopropyl Myristate | 8 ± 3 |
| Ethyl Oleate | 7 ± 3 |
| Propylene Glycol | 5 ± 2 |
| Span 20 | 3 ± 1 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of diclofenac acid from the above non-volatile solvents are obtained by placing 200 mcL on the stratum corneum side (donor) of hairless mouse skin. The in vitro studies are carried out as described in Example 1. From Table 4, the non-volatile solvent glycerol has no reported steady state flux value and is not considered a flux enabling non-volatile solvent viable non-volatile solvent candidate. However, the steady state flux values of isopropyl myristate, ethyl oleate, propylene glycol, and Span 20 are no more than 10 times the flux value reported for currently available marketed products, and as such, could be considered flux-enabling solvents. It should be noted that the steady state flux values for diclofenac acid from each of the above non-volatile solvents are much lower than the steady state flux values obtained with diclofenac sodium. Therefore, if therapeutically sufficient flux values need to be increased, utilizing a flux-enabling non-volatile solvent and the salt form of diclofenac would likely yield higher steady state flux values than using the acid form of diclofenac.

### Examples 5-7

Prototype peel formulations are prepared as follows. Several peel formulations are prepared in accordance with embodiments of the present invention in accordance with Table 5, as follows:

TABLE 5

| Example | | |
|---|---|---|
| 5 | 6 | 7 |

% by weight

Volatile Solvents

| | | | |
|---|---|---|---|
| Ethanol | 21 | 24 | 18.5 |
| Water | 32 | | 28 |

Solidifying agents

| | | | |
|---|---|---|---|
| Eudragit RL-PO | | 40 | |
| Eudragit E-100 | | | 18.5 |
| Polyvinyl Alcohol | 21 | | 18.5 |

Non-volatile solvents

| | | | |
|---|---|---|---|
| Glycerol | | 12 | |
| Propylene Glycol | 21 | 4 | |
| Isostearic Acid | | 13 | |
| Span 20 | | | 11 |
| Trolamine | | 4 | |

Drug

| | | | |
|---|---|---|---|
| Ketoprofen | 5 | | |
| Ropivacaine | | 3 | |
| Diclofenac Na | | | 5.5 |

Peel formulations of Examples 5-7 are prepared in the following manner:
The solidifying agents are dissolved in the volatile solvent (e.g., dissolve polyvinyl alcohol in water, Eudragit polymers in ethanol),
The non-volatile solvent is mixed with the solidifying agent/volatile solvent mixture.
The resulting solution is vigorously mixed well for several minutes.
The drug is then added and the peel formulation is mixed again for several minutes.

In all the examples noted above, the flux-enabling non-volatile solvent/solidifying agent/volatile solvent combination is compatible as evidenced by a homogeneous, single phase system that exhibited appropriate drying time, and provided a stretchable peel and steady state flux for the drug (see Example 8 below).

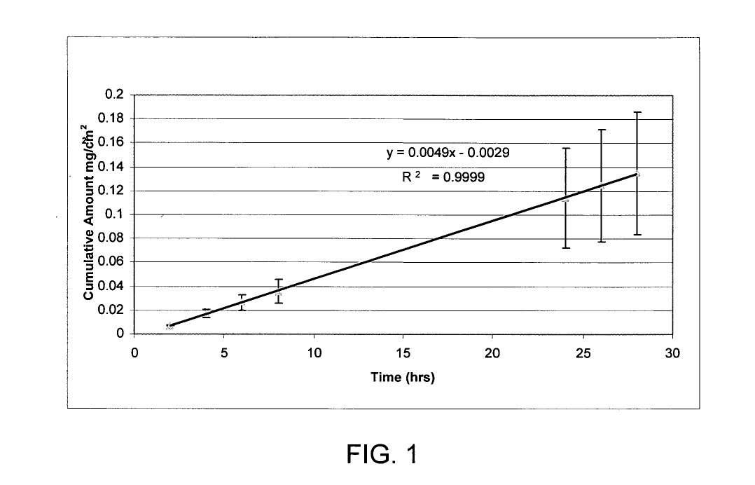

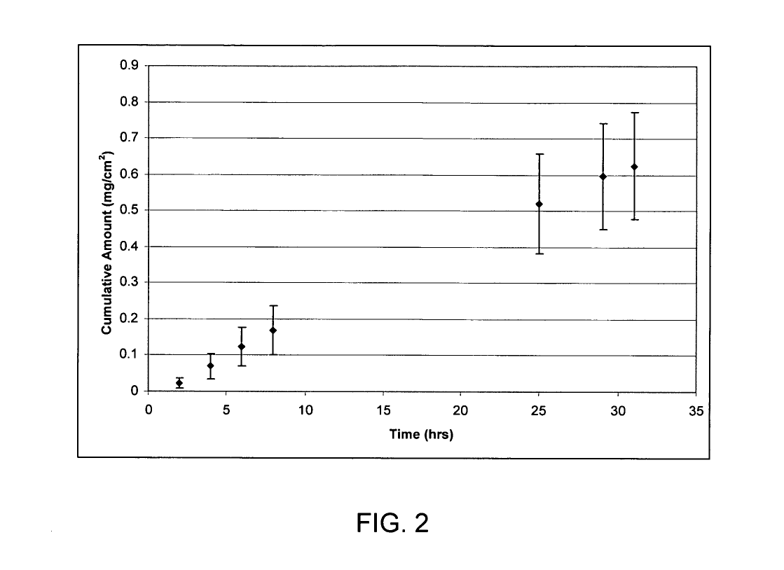

FIGS. 1 and 2 provide a graphical representation of the cumulative amount of diclofenac and ropivacaine, respectively, delivered transdermally across human cadaver skin. The formulations tested were similar to those described in Examples 6 and 7. In these particularly embodiments, steady-state delivery is shown over 28 hours, and over 30 hours, respectively.

### Example 8

The formulations of the examples are tested in a hairless mouse skin (HMS) or human epidermal membrane (HEM) in vitro model described in Example 1. Table 6 shows data obtained using the experimental process outlined above.

TABLE 6
Steady-state flux (J)

| | J* |
|---|---|
| Formulation | (μg/cm²/h) |
| Example 5 | 35 ± 20*** |
| Example 6 | 32 ± 2*** |
| Example 7 | 5 ± 2**** |

*Skin flux measurements represent the mean and standard deviation of three determinations.
***Flux measurements across HMS reported were determined from the linear region of the cumulative amount versus time plots. The linear region was observed to be between 4-8 hours. If experimental conditions allowed, the steady-state delivery would likely continue well beyond 8 hours.
****Flux measurements across HEM reported were determined from the linear region of the cumulative amount versus time plots. The linear region was observed to be between 6-28 hours. If the experiment was continued it is anticipated the steady state would continue.

In all cases in Table 6, the flux enabling non-volatile solvents in the formulation resulted in therapeutically sufficient flux for each of the formulations studied.

### Example 9

A placebo formulation with the following composition: 10.4% polyvinyl alcohol, 10.4% polyethylene glycol 400, 10.4% polyvinyl pyrrolidone K-90, 10.4% glycerol, 27.1% water, and 31.3% ethanol was applied onto a human skin surface at an elbow joint and a finger joint, resulting in a thin, transparent, flexible, and stretchable film. After a few minutes of evaporation of the volatile solvents (ethanol and water), a solidified layer that was peelable was formed. The stretchable peel had good adhesion to the skin and did not separate from the skin on joints when bent, and could easily be peeled away from the skin. Addition of an active drug into this placebo formulation is not expected to significantly change the physical properties of the initial formulation or the solidified layer, as the concentration of the active drug as a percentage of the total weight of the formulation is typically small.

### Examples 10-12

Three formulations are applied on the stratum corneum side of freshly separated hairless mouse skin. The in vitro flux is determined for each formulation as outlined in Example 1. The formulation compositions are noted in Table 7 below.

TABLE 7

| | Example | | |
|---|---|---|---|
| | 10 | 11 | 12 |
| | % by weight | | |
| PVA | 15 | 15 | 15 |
| Water | 23 | 23 | 23 |
| Ethylcellulose ECN-100 | 11 | 11 | 11 |
| Ethanol | 33 | 33 | 33 |
| Span 20 | 11 | | |
| Polyethylene Glycol 400 | | 11 | |
| Tween 40 | | | 11 |
| Tromethamine | 4 | 4 | 4 |
| Ropivacaine HCl | 3 | 3 | 3 |
| Avg. Flux* (mcg/cm²/h) | 15 ± 1 | 4.7 ± 0.3 | 3.4 ± 0.7 |

*Flux values represent the mean and standard deviation of three determinations. Flux measurements reported were determined from the linear region of the cumulative amount versus time plots. The linear region was observed to be between 4-9 hours. If the experiment was continued it is anticipated the steady state would continue.

Since all three formulations have the exact same compositions of solidifying agent, volatile solvents, and flux-enabling non-volatile solvent. The only difference is which flux-enabling non-volatile solvent is used it is reasonable to conclude that for ropivacaine HCl that Example 10 is flux enabling.

### Examples 13-14

A peel-forming formulation for dermal delivery of ropivacaine is prepared which includes a specified amount of ropivacaine in an excipient mixture to form an adhesive formulation in accordance with embodiments of the present invention. The peel formulations contained the following components:

TABLE 8
Ropivacaine peelable formulations

Examples

| Ingredients* | 13 | 14 |
|---|---|---|
| Eudragit RL-100 | 39.6% | 39.6% |
| Ethanol | 23.7% | 23.6% |
| ISA (Isostearic Acid) | 13.5% | 13.5% |
| PG (Propylene Glycol) | 7.9% | 4.0% |
| Trolamine | 4.0% | 4.0% |
| Glycerol | 7.9% | 11.9% |
| Ropivacaine | 3.4% | 3.4% |

*Ingredients are noted as weight percent.

These formulations are applied to HMS skin as described in Example 1, and the ropivacaine flux is measured. A summary of the results from in vitro flux studies carried out with the formulations in Examples 13 and 14 is listed in Table 9.

TABLE 9
Steady-state flux of ropivacaine through hairless mouse skin
from various adhesive peelable formulations at 35° C.

| | Average flux |
|---|---|
| Formulation | mcg/cm²/h* |
| Example 13 | 36 ± 5 |
| Example 14 | 32 ± 2 |

*The flux values represent the mean and SD of three determinations

Regarding the formulation described in Examples 13 and 14, ethanol is used as the volatile solvent, and the ISA, glycerol, and PG mixture is used as the non-volatile solvent system. Through experimentation, it is determined that ISA and propylene glycol used together to provide the appropriate flux for the drug, while being compatible with the Eudragit RL-100 solidifying agent. Further, in this embodiment, ISA, PG and glycerol serve as a plasticizer in the peelable formulation after the ethanol (volatile solvent) has evaporated. The steady state flux of ropivacaine from formulation Examples 13 and 14 demonstrate the importance of the non-volatile solvent in dictating the flux-generating power of the entire formulation.

### Example 15

The effect of solubility on permeation, compatibility between the non-volatile solvent system and the solidifying agent is shown in this Example.

Ropivacaine base solubility in isostearic acid (ISA) is experimentally determined to be slightly above 1:4, meaning 1 gram ropivacaine base can completely dissolve in 4 gram isostearic acid. In one experiment, two solutions are made: Solution A includes 1 part ropivacaine base and 4 parts isostearic acid. Solution B includes 1 part ropivacaine base, 4 parts isostearic acid, and 1 part trolamine. (all parts are in weight). All ropivacaine in Solution A is dissolved, but only a portion of ropivacaine in solution B is dissolved. The transdermal flux across hairless mouse skin generated by the solutions is measured by a typical Franz Cell system, with the following results:

TABLE 10
Flux across hairless mouse skin, in vitro, in μg/hr/cm²

| | Cell 1 | Cell 2 | Cell 3 | Average |
|---|---|---|---|---|
| Solution A | 13.1 | 9.9 | 9.1 | 10.7 |
| Solution B | 43.2 | 35.0 | 50.0 | 42.7 |

As can be seen, the flux generated by Solution B is about 4 times that of Solution A. These results demonstrate that the addition of the ion paring agent trolamine significantly increases the transdermal flux. However, the attempt to incorporate this system into a poly vinyl alcohol (PVA) based peel formulation failed because the PVA in the formulation acted as a strong pH buffer that inhibited the effect of trolamine. Addition of more trolamine, in attempt to over-power the pH buffer capacity of PVA, caused the loss of the desired solidifying property of PVA (in other words, a non-volatile solvent system containing ISA and too much trolamine is not compatible with PVA). When PVA is replaced by another solidifying agent, Eudragit RL 100 (Rohm & Haas), the effect of trolamine is not inhibited and formulations capable of generating fluxes around 30 μg/hr/cm² were obtained. A by product of the addition of trolamine, ISA, and Eudragit RL 100 is that a precipitate forms from the ionic interaction of the three components. The latter Example produced a better formulation in terms of flux and wear properties, but the precipitation still demonstrates the need for improvement. In an effort to eliminate the ionic interaction between non-volatile solvent and solidifying agent the trolamine, ISA mixture was added to Plastoid B polymer in isopropanol. However, in this instance the trolamine was found to be incompatible with the Plastoid B polymer and the base was changed to triisopropanolamine. This combination eliminated the precipitate formed when the Eudragit RL 100 polymer was used and produced a clear formulation that was capable of generated flux values around 30 μg/hr/cm². This demonstrates the importance of compatibility between the non-volatile solvent system and the solidifying agent.

### Example 16

A solidifying formulation for dermal delivery of ropivacaine is prepared from the following ingredients:

TABLE 11
Ropivacaine solidifying formulation components

| | Example |
|---|---|
| Ingredients* | 16 |
| Ropivacaine HCl | 0.096 |
| Eudragit RL-100 | 1.0 |
| Ethanol | 0.7 |
| Isostearic Acid | 0.34 |
| Glycerol | 0.3 |
| Propylene Glycol | 0.1 |
| Trolamine | 0.15 |

*Ingredients are noted as parts by weight.

The ingredients listed above are combined according to the following procedure. The Eudragit RL-100 and ethanol are combined in a glass jar and heated to about 60° C. until the Eudragit RL-100 is completely dissolved. Once the Eudragit solution cooled to room temperature, the appropriate amount of ropivacaine HCl is added and mixed thoroughly for 1 minute. To this solution, isostearic acid (ISA) is added and the mixture is stirred vigorously for 2-3 minutes. One hour later, the solution is vigorously mixed again for 2-3 minutes. To this solution, glycerol, propylene glycol, and trolamine are added in sequential order. After addition of each ingredient the solution is stirred for 1 minute.

### Example 17

The formulation prepared in accordance with Example 16 is applied to HMS as described in Example 1, and the ropivacaine flux was measured. A summary of the results is listed in Table 12, as follows:

TABLE 12
Steady-state flux of ropivacaine through hairless mouse skin
from various adhesive peelable formulations at 35° C.

| | Average flux |
|---|---|
| Formulation | mcg/cm²/h* |
| Example 16 | 43 ± 4 |

*The flux values represent the mean and SD of three determinations

The ropivacaine peel formulation prepared in accordance with Example 16 possessed acceptable application properties, e.g., ease of removal of peel from the sample tube, ease of spreading on intended skin application site, etc., and forms a solidified film in 2-3 minutes after being applied to normal human skin surface as a thin layer with a thickness of about 0.1 mm. The solidified layer becomes more easily peelable in 2 hours, and the peel remains affixed to the skin surface without any unintended removal of the peel for at least 12 hours. At the end of intended use, the peel is easily removed in one continuous piece.

### Example 18

A solidifying formulation for dermal delivery of lidocaine (base) is prepared which includes a saturated amount of lidocaine in an excipient mixture to form an adhesive formulation in accordance with embodiments of the present invention. The peel formulation is prepared from the ingredients as shown in Table 13.

TABLE 13
Lidocaine solidifying formulation components.

| | Example |
|---|---|
| Ingredients* | 18 |
| PVA | 11.7 |
| Eudgragit E-100** | 11.7 |
| PVP-K90 | 5.8 |
| Glycerol | 8.8 |
| PEG-400 | 8.8 |
| Water | 23.8 |
| Ethanol | 23.8 |
| Lidocaine | 5.6 |

*Ingredients are noted as weight percent.
**from Rohm & Haas.

TABLE 14
Steady-state flux of lidocaine through hairless mouse skin from
various adhesive solidifying formulations at 35° C.

| | Average flux |
|---|---|
| Formulation | mcg/cm²/h* |
| Example 18 | 47 ± 3 |

The adhesive formulation of lidocaine formulation in the present Example 18 has similar physical properties to the examples noted above. The transdermal flux across hairless mouse skin is acceptable and steady-state delivery is maintained over 8 hours.

### Examples 19-22

Solidifying formulations for dermal delivery of ropivacaine are prepared which includes an excipient mixture to form an adhesive solidifying formulation in accordance with embodiments of the present invention. The peel formulations are prepared from the ingredients as shown in Table 15.

TABLE 15
Ropivacaine HCl solidifying formulation components.

Example

| Ingredients* | 19 | 20 | 21 | 22 |
|---|---|---|---|---|
| Ropivacaine HCl | 0.31 | 0.31 | 0.31 | 0.31 |
| Isopropanol | 2 | 2 | 2.2 | 2 |
| Water | 0.125 | 0.125 | 0.125 | 0.125 |
| Isostearic Acid | 0.36 | 0.66 | 0.41 | 0 |
| Triisopropanolamine | 0.31 | 0.34 | 0.34 | 0.34 |
| Triacetin | 0.17 | 0.19 | 0 | 0.19 |
| Span 20 | 0.34 | 0 | 0.37 | 0.66 |
| Plastoid B** | 1 | 1 | 1 | 1 |

*Ingredients are noted as parts by weight.
**from Degussa.

The ingredients listed above are combined according to the following procedure. The ropivacaine HCl, water, and triisopropanolamine are combined in a glass jar and mixed until the drug is dissolved. Then the isostearic acid, triacetin, Span 20, and isopropanol are added to the formulation and mixed well. The polymer Plastoid B is added last and heated to about 60° C. until the Plastoid B is completely dissolved. Once the polymer solution cooled to room temperature, the formulation is stirred vigorously for 2-3 minutes.

The formulations in Table 15 are applied to HMS according to Example 1, and the flux of ropivacaine was measured. The results are summarized in Table 16:

TABLE 16
Steady-state flux of ropivacaine HCl through hairless mouse skin
from various adhesive solidifying formulations at 35° C.

| | Average flux |
|---|---|
| Formulation | mcg/cm²/h* |
| Example 19 | 56 ± 2 |
| Example 20 | 39 ± 6 |
| Example 21 | 31 ± 6 |
| Example 22 | 37 ± 9 |

The flux of Examples 19-22 show the importance of the triacetin, isostearic acid, Span 20 combination in the formulation. In Examples 20-22 formulations were made without Span 20, triacetin, and isostearic acid respectively. The in vitro flux of ropivacaine was impacted. The synergistic combination of the non volatile solvents is an important in obtaining the maximum in vitro flux of ropivacaine.

### Example 23

This solidifying formulation has the following ingredients in the indicated weight parts:

TABLE 17

| | | Ethyl | Dermacryl | | | | |
|---|---|---|---|---|---|---|---|
| | | Cellulose | 79 | | Isostearic | | |
| | | ECN-7 | (National | | Acid | | |
| PVA | Water | (Aqualon) | Starch) | Ethanol | (ISA) | Glycerol | Ropivacaine |
| 1 | 1.5 | 0.25 | 0.35 | 0.85 | 0.8 | 0.35 | 0.3 |

In this formulation, polyvinyl alcohol (USP grade MW 31,000-50,000, from Amresco) is a solidifying agent, ethyl cellulose and Dermacryl 79 are auxiliary solidifying agents. Isostearic acid and glycerol form the non-volatile solvent system while ethanol and water form the volatile solvent system. Ropivacaine is the drug.
Procedures of making the formulation:
1. Ropivacaine is mixed with ISA.
2. Ethyl cellulose and Dermacryl 79 are dissolved in ethanol.
3. PVA is dissolved in water at temperature of about 60-70 C.
4. All of the above mixtures are combined together in one container and glycerol is added and the whole mixture is mixed well.
   The resulting formulation is a viscous fluid. When a layer of about 0.1 mm thick is applied on skin, a non-tacky surface is formed in less than 2 minutes.

### Examples 24-27

A stretchable adhesive formulation for transdermal delivery of ketoprofen (which is suitable for delivery via skin for treating inflammation or pain of joints and muscles) is prepared which includes saturated amount of ketoprofen in an excipient mixture (more ketoprofen than that can be dissolved in the excipient mixture) to form an adhesive formulation, some of which is prepared in accordance with embodiments of the present invention. The excipient mixture, which is a viscous and transparent fluid, is prepared using the ingredients as shown in Table 18.

TABLE 18
Ketoprofen solidifying formulation components

| | Examples | | | |
|---|---|---|---|---|
| Ingredients* | 24 | 25 | 26 | 27 |
| PVA (Polyvinyl Alcohol) | 10.4 | 21.4 | 21.1 | 21.2 |
| PEG-400 (Polyethylene Glycol) | 10.4 | 10.8 | 2.9 | 18.6 |
| PVP-K90 (Polyvinyl Pyrrolidone) | 10.4 | 0.0 | 0.0 | 0.0 |
| Glycerol | 10.4 | 10.8 | 19.0 | 2.9 |
| Water | 27.1 | 57.0 | 57.0 | 57.3 |
| Ethanol | 31.3 | 0 | 0 | 0 |
| Ketoprofen | satu- | satu- | satu- | satu- |
| | rated | rated | rated | rated |

*Ingredients are noted as % by weight.

Each of the compositions of Examples 24-27 were studied for flux of ketoprofen, as shown in Table 19, as follows:

TABLE 19
Steady-state flux of Ketoprofen through hairless mouse
skin from various adhesive formulations at 35° C.

| | Average flux |
|---|---|
| Formulation | mcg/cm²/h* |
| Example 24 | 8 ± 3 |
| Example 25 | 21 ± 6 |
| Example 26 | 3 ± 1 |
| Example 27 | 1 ± 0.4 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

Regarding formulation described in Example 24, ethanol and water formed the volatile solvent system, while a 1:1 mixture of glycerol and PEG 400 formed the non-volatile solvent system. Through experimentation, it is determined that PEG 400 is a slightly better solvent than glycerol for ketoprofen, while glycerol is much more compatible with PVA than PEG 400. Thus, the non-volatile solvent system of glycerol and PEG 400 are used together to provide a non-volatile solvent system for the drug, while being reasonably compatible with PVA. In additional detail with respect to the formulation in Example 24, PVA and PVP act as the solidifying agents. Further, in this embodiment, glycerol and PEG 400 also serve as plasticizers in the adhesive formulation formed after the evaporation of the volatile solvents. Without the presence of glycerol and PEG 400, a film formed by PVA and PVP alone would be rigid and non-stretchable.

Regarding the formulation of Example 25, the adhesive peelable formation formed has similar physical properties as that of Example 24, though the transdermal flux across hairless mouse skin is higher. This suggests that the solidifying agent, 1:1 PVA:PVP-K-90 in Example 24 and pure PVA in Example 25, have an impact on permeation.

The formulation in Example 26 delivers less ketoprofen than the formulations of Examples 24 or 25 The formulation of Example 27 delivers much less ketoprofen than the formulations in Examples 24 and 25. One possible reason for the reduced flux is believed to be the reduced permeation driving force caused by the high concentration of PEG 400 in the non-volatile solvent system, which resulted in too high of solubility for ketoprofen.

The only significant difference among the formulations in Examples 25, 26, and 27, respectively, is with respect to the non-volatile solvent system, or more specifically, the PEG 400:glycerol weight ratio. These results reflect the impact of the non-volatile solvent system on skin flux.

### Example 28

A stretchable adhesive formulation for transdermal delivery of ketoprofen (which is suitable for delivery via skin for treating inflammation or pain of joints and muscles) is prepared which includes ketoprofen in an excipient mixture to form an adhesive formulation, some of which is prepared in accordance with embodiments of the present invention. The peel formulation is prepared from the ingredients as shown in Table 20.

TABLE 20
Ketoprofen solidifying formulation components

| | Example | Example |
|---|---|---|
| Ingredients* | 29 | 30 |
| PVA | 22.1 | 18.9 |
| Water | 30.9 | 37.9 |
| Fumed Silica | | 3.0 |
| Glycerol | 11.1 | 9.5 |
| Propylene glycol | 17.7 | 15.2 |
| Gantrez ES-425 | 4.4 | 3.8 |
| Ethanol | 8.8 | 7.6 |
| Ketoprofen | 5.0 | 4.2 |

*Ingredients are noted as weight percent.

TABLE 21
Steady-state flux of Ketoprofen through hairless mouse skin
from an adhesive solidifying formulations at 35° C.

| | Average flux |
|---|---|
| Formulation | mcg/cm²/h* |
| Example 29 | 25 ± 6 |
| Example 30 | 27 ± 2 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

### Examples 29-31

A stretchable adhesive formulation for transdermal delivery of ketoprofen (which is suitable for delivery via skin on joints and muscles) is prepared which includes saturated amount of ketoprofen in an excipient mixture (more ketoprofen than that can be dissolved in the excipient mixture) to form an adhesive formulation, some of which are prepared in accordance with embodiments of the present invention. The excipient mixture, which is a viscous and transparent fluid, is prepared using the ingredients as shown in Table 22.

TABLE 22

| | Examples | | |
|---|---|---|---|
| Ingredients* | 29 | 30 | 31 |
| Eugragit RL-PO | 28.06 | 27.7 | 27.5 |
| Ethanol | 40.07 | 39.5 | 39.5 |
| Glycerol | 27.40 | 13.9 | |
| Polyethylene Glycol 400 (PEG) | | 13.9 | 28. |
| Ketoprofen | 4.5 | 5 | 5 |

Peel formulations of Examples 29-31 are prepared in the following manner:
The solidifying agents are dissolved in the volatile solvent (i.e., dissolve Eudragit polymers in ethanol).
The flux adequate non-volatile solvent (glycerol, PEG) is mixed together with the solidifying agent/volatile solvent mixture.
The resulting solution is vigorously mixed for several minutes.
Drug is then added and the formulation is mixed again for several minutes.

### Example 32

The formulations prepared in accordance with Example 29-31 are applied to HMS as described in Example 1, and the ketoprofen flux is measured. A summary of the results is listed in Table 23, as follows:

TABLE 23
Steady-state flux of ketoprofen through hairless mouse skin

| | Average flux |
|---|---|
| Formulation | mcg/cm²/h* |
| Example 29 | 15 ± 7 |
| Example 30 | 10 ± 3 |
| Example 31 | 4 ± 1 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

The ketoprofen adhesive solidifying formulations prepared in accordance with Examples 29-30 possessed acceptable solidified film properties (e.g., formed a solidified layer in 2-3 minutes). With Example 31, the ketoprofen formulation does not form a solidified layer 30 minutes after application. This demonstrates that order to obtain desired flux and wear properties in a peel formulation, a delicate balance between solidifying agents, non-volatile solvents, and volatile solvents is evaluated and considered in developing a formulation.

### Example 33

An adhesive solidifying formulation for transdermal delivery of ketoprofen, which can form elastic solidified layers and is suitable for delivery via skin on joints and muscles, is prepared which includes saturated amount of ketoprofen in an excipient mixture (more ketoprofen than that can be dissolved in the excipient mixture) to form an adhesive formulation, some of which are prepared in accordance with embodiments of the present invention. The excipient mixture, which is a viscous and transparent fluid, is prepared using the ingredients as shown in Table 24.

TABLE 24

| | FORMULATIONS | | |
|---|---|---|---|
| Ingredients* | A | B | C |
| PVA (Celvol 502 MW 10,000) | 24.4 | | |
| PVA (Amresco MW 31,000-50,000) | | 24.4 | |
| PVA (Celvol 523 MW 125,000) | | | 41.7 |
| Water | 33.4 | 33.4 | 58.3 |
| Ethanol | 8.9 | 8.9 | |
| PG | 17.8 | 17.8 | |
| Glycerol | 11.1 | 11.1 | |
| Gantrez ES 425 | 4.4 | 4.4 | |

*Ingredients are noted in weight percent.

Formulations A and B are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.
   Preparation of the PVA in water solution in Formulation C was not feasible for this molecular weight of PVA at the percentages noted. Formulation C demonstrates that the correct polymer molecular weight for PVA is important to obtain the desired formulation properties.

Formulations A and B are placed on the skin of human volunteers. After a period of several hours, long enough for the volatile solvent to evaporate, the peels were removed by the volunteers and the peelability properties were evaluated. In all instances the volunteers reported that formulation example A could not be removed in one or two pieces, but was removed in numerous small pieces. Formulation example B removed in one or two pieces. The lack of cohesion nature of formulation A is attributed to the lower molecular weight PVA sample (Celvol). Low molecular weight PVA does not possess the same cohesive strength as higher molecular weight PVA material (Amresco) due to the reduced size of the polymer chain leading to a reduction in the degree of cross linking and physical interactions between individual PVA polymer chains. The reduced PVA chain interactions lead to a weaker solidified layer that is unable to withstand the mechanical forces it is subjected to upon removal.

### Examples 34-35

A stretchable adhesive formulation for transdermal delivery of ketoprofen (which is suitable for delivery via skin on joints and muscles) was evaluated which includes a placebo excipient mixture which will form an adhesive formulation, some of which are prepared in accordance with embodiments of the present invention. The excipient mixture, which is a viscous and transparent fluid, is prepared using the ingredients as shown in Table 25.

TABLE 25

Examples

| Ingredients* | 34 | 35 |
|---|---|---|
| PVA (Amresco MW 31,000-50,000) | 20.41 | 21.28 |
| Water | 30.61 | 27.66 |
| Ethanol | 20.41 | 21.28 |
| PG | 20.41 | 21.28 |
| Glycerol | 6.12 | 6.38 |
| Gantrez S97 | 2.04 | 2.13 |

*Ingredients are noted in weight percent.

Peel formulations in Examples 1 and 2 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez S97 is added to the mixture.
The resulting solution is vigorously mixed for several minutes.

Formulations above were applied on the forearms of study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the peel from the skin or leaving residue behind. The time when this observation is made is defined as the drying time for the peel formulation. The results of the study are summarized in Table 26 below.

TABLE 26

| Example | Drying Time (min) |
|---|---|
| 34 | 7.0 |
| 35 | 6.5 |

The amount of water in the formulation did not significantly influence the time for the formulation to dry. However, it was noted during the study that the formulation was difficult to expel from the sample tube. After approximately 4 weeks after the formulation in examples 1 and 2 were made the sample tubes were retrieved and were evaluated for ease of dispensing the formulation. It was noted that the formulation was impossible to expel from the tube. Interpolymer complexation between Gantrez S-97 and PVA through electrostatic interactions, hydrophobic interactions, hydrogen bonding, or Van der Waals interactions is hypothesized to be the reason(s) for the observed thickening. Moreover, the extent of this interaction may be dependent on the stoichiometric ratio of the two polymers. It is believed that the water content of the formulations is too low for obtaining acceptable long term physical stability, although the formulation shorter term viscosity was acceptable. This demonstrates the value of having sufficient amount of the volatile solvent system in the formulation in some embodiments.

### Examples 36-39

A stretchable adhesive formulation for transdermal delivery of ketoprofen (which is suitable for delivery via skin on joints and muscles) was evaluated which includes an excipient mixture which will form an adhesive formulation, some of which are prepared in accordance with embodiments of the present invention. The excipient mixture, which is a viscous and transparent fluid, is prepared using the ingredients as shown in Table 27.

TABLE 27

Examples

| Ingredients* | 36 | 37 | 38 | 39 |
|---|---|---|---|---|
| PVA (Amresco MW | 22.1 | 24.4 | 22.1 | 21.1 |
| 31,000-50,000) | | | | |
| Water | 26.6 | 29.2 | 30.9 | 33.8 |
| Ethanol | 12.6 | 4.2 | 8.4 | 8.2 |
| Butanol | 0.4 | 0.5 | 0.4 | 0.4 |
| PG | 19.9 | 21.9 | 17.7 | 16.9 |
| Glycerol | 8.8 | 9.7 | 11 | 10.6 |
| Gantrez ES 425 | 4.6 | 5.1 | 4.4 | 4.0 |
| Ketoprofen | 5.0 | 5.0 | 5.1 | 5.0 |

*Ingredients are noted in weight percent.

Peel formulations in Examples 1-4 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.
After mixing, ketoprofen is added and the final mixture is vigorously mixed again for several minutes.

Formulations noted above were placed in laminate packaging tubes and stored at 25 C/60% RH and 40 C/75% RH conditions until pulled for testing. Physical testing was performed on each formulation. Table 28 summarizes the data generated on each formulation.

TABLE 28

| | Viscosity* | | | | | |
|---|---|---|---|---|---|---|
| Example | cPs | | | | | |
| Storage Cond. | T = 0 | 2 weeks | 4 weeks | 8 weeks | 12 weeks | 16 weeks |
| 36 | 96000 | 670000 | >2500000 | Not | | |
| 25 C./60% RH | | | | measured | | |
| 36 | 96000 | 500000 | 587500 | 2320000 | | |
| 40 C./75% RH | | | | | | |
| 37 | 168500 | 204500 | 251000 | >2500000 | | |
| 25 C./60% RH | | | | | | |
| 37 | 168500 | 215000 | 217500 | >2500000 | | |
| 40 C./75% RH | | | | | | |
| 38 | 23000 | — | 25000 | 36250 | 76250 | 57500 |
| 25 C./60% RH | | | | | | |
| 38 | 23000 | — | 31000 | 40000 | 243500 | 164500 |
| 40 C./75% RH | | | | | | |
| 39 | 11250 | 13750 | | | | |
| 25 C./60% RH | | | | | | |
| 39 | 11250 | 17500 | | | | |
| 40 C./75% RH | | | | | | |

*Viscosity measured using a RVDV 1+ viscometer at 0.5 rpm.

Examples 36 and 37 had the lowest water content of the four formulations and within 4 weeks of storage attained high viscosity values. The only difference between Examples 36 and 37 is the amount of ethanol in the formulations. It was hypothesized that reducing the level of ethanol may reduce the physical thickening of the formulation due to an incompatibility between the PVA and ethanol. The viscosity data show that the higher ethanol formulation (Example 36) had lower initial viscosity, but over the 4 weeks storage the viscosity of both Example 36 and 37 attained viscosity values that were too high for a viable formulation. Another hypothesis for the formulation thickening is that PVA is not compatible in high concentrations when dissolved in water. Additional formulations with higher water content were prepared to determine if an optimal water amount would keep the formulation from thickening up over time. Example 38 viscosity after 16 weeks has not reached the viscosity values of the initial viscosity values of Examples 36 and 37.

Placebo versions of the formulations above were applied on study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the peel or leaving residue behind. The results of the study are summarized in Table 29 below.

TABLE 29

| Example | Drying Time (min)* |
|---|---|
| 36 | 4 min 49 sec |
| 37 | 5 min 41 sec |
| 38 | 4 min 27 sec |
| 39 | 5 min 1 sec |

*average dry time value from 12 study subjects.

The presence of ethanol as a second volatile solvent appears to significantly reduce the time to dry. In data not shown a local anesthetic formulation containing only water as the volatile solvent and a ratio of water to PVA of 2:1 has a drying time of >15 minutes. Optimizing the ratio and the presence of an additional volatile solvent in formulations containing water significantly reduce the drying time. It is hypothesized that the additional volatile solvent, in this case ethanol, will hydrogen bond with the water and water will escape with the ethanol when evaporating off the skin thereby forming a solidified layer. This example demonstrates the value of using the right mixture and quantities of volatile solvents in the volatile solvent system in certain embodiments.

### Examples 40-42

Solidifying formulations for dermal delivery of ropivacaine HCl are prepared which include excipient mixtures in accordance with embodiments of the present invention. The formulations are prepared from the ingredients as shown in Table 30.

TABLE 30
Ropivacaine HCl solidifying formulation components.

| | Example | | |
|---|---|---|---|
| Ingredients* | 40 | 41 | 42 |
| Ropivacaine HCl | 6.9 | 6.5 | 6.6 |
| Isopropanol | 50.7 | 45.8 | 45.9 |
| Water | 5.5 | 5.2 | 5.2 |
| Isostearic Acid | 6.3 | 6.6 | 6.6 |
| Triethylamine | | | 3.0 |
| Diisopropanolamine | | 3.9 | |
| Cetyl alcohol | | 3.3 | 3.9 |
| Triacetin | 2.9 | 2.6 | 2.6 |
| Span 20 | 5.8 | 5.2 | 5.2 |
| Plastoid B** | 21.9 | 20.9 | 21.0 |

*Ingredients are noted as weight percent.
**from Degussa.

The ingredients listed above are combined according to the following procedure. The ropivacaine HCl, water, and the amine base (triethylamine or diisopropanolamine) are combined in a glass jar and mixed until the drug is dissolved. Then the isostearic acid, triacetin, Span 20, and cetyl alcohol (Examples 41 and 42), or isopropanol (Example 40) are added to the formulation and mixed well. The polymer Plastoid B is added last and heated to about 60° C. until the Plastoid B is completely dissolved. Once the polymer solution cooled to room temperature, the formulation is stirred vigorously for 2-3 minutes.

The formulations in Table 30 are applied to HMS according to Example 1, and the flux of ropivacaine was measured. The results are summarized in Table 31:

TABLE 31
Steady-state flux of ropivacaine HCl through hairless mouse skin
from various adhesive solidifying formulations at 35° C.

| | Average flux |
|---|---|
| Formulation | mcg/cm²/h* |
| 40 | 96 ± 14 |
| 41 | 61 ± 2 |
| 42 | 70 ± 7 |

### Example 43

Solvent formulations of ketoprofen in various non-volatile solvent systems are evaluated. Excess ketoprofen is present.

The permeation of ketoprofen from the test formulations through HMS is presented in Table 32 below.

TABLE 32

| | Skin Flux* |
|---|---|
| Non-volatile solvent system | (mcg/cm²/h) |
| Glycerol | 2 ± 1 |
| Polyethylene Glycol 400 | 5 ± 2 |
| Span 20 | 15 ± 3 |
| Propylene Glycol | 90 ± 50 |
| Oleic Acid | 180 ± 20 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of ketoprofen from the above non-volatile solvents are obtained by placing 200 mcL on the stratum corneum side (donor) of hairless mouse skin. The in vitro studies are carried out as described in Example 1. From Table 32, the non-volatile solvents glycerol and polyethylene glycol 400 had low steady state flux values and would not be considered “flux-enabling.” Span 20 maybe considered flux-enabling, and propylene glycol or oleic acid provided the highest flux and are considered flux-enabling non-volatile solvent systems. Assessment of flux-enabling solvents is based on the estimated therapeutically sufficient flux of 16 mcg/cm²/h for ketoprofen. Steady state flux values of a drug from the non-volatile solvent that are below the therapeutically sufficient flux are not considered flux-enabling while steady state flux values of a drug from a non-volatile solvent above the therapeutically sufficient flux value is considered flux-enabling.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A formulation for treating musculoskeletal pain or inflammation, comprising:
a) a drug suitable for treating musculoskeletal pain or inflammation;
b) a solvent vehicle, comprising:
i) a volatile solvent system including at least one volatile solvent, and
ii) a non-volatile solvent system including at least one non-volatile solvent, and
c) a solidifying agent,
wherein the formulation has a viscosity suitable for application and adhesion to a skin surface as a layer prior to evaporation of the volatile solvent system, the layer applied to the skin surface forms a solidified layer after at least partial evaporation of the volatile solvent system, and the drug continues to be dermally delivered at the therapeutically effective rate to treat musculoskeletal pain or inflammation after the volatile solvent system is at least substantially evaporated.

2. A formulation as in claim 1, wherein the non-volatile solvent system acts as a plasticizer for the solidifying agent.

3. A formulation as in claim 1, wherein the non-volatile solvent system facilitates transdermal delivery of the drug at a therapeutically effective rate over a sustained period of time.

4. A formulation as in claim 1, wherein the non-volatile solvent system is flux-enabling for the drug.

5. A formulation as in claim 1, wherein the formulation further comprises a pH modifying agent

6. A formulation as in claim 1, wherein the musculoskeletal pain or inflammation is in or around a finger joint.

7. A formulation as in claim 1, wherein the musculoskeletal pain or inflammation is in or around a wrist, elbow, or knee.

8. A formulation as in claim 1, wherein the musculoskeletal pain or inflammation is in or around a back or neck.

9. A formulation as in claim 1, wherein the formulation further comprises an additional agent which is added to increase adhesion of the formulation when applied to the skin surface.

10. A formulation as in claim 9, wherein the additional agent includes at least one member selected from the group consisting of copolymers of methylvinyl ether and maleic anhydride, polyethylene glycol and polyvinyl pyrrolidone, gelatin, low molecular weight polyisobutylene rubber, copolymer of acrylsan alkyl/octylacrylamido, aliphatic resins, aromatic resins, and combinations thereof.

11. A formulation as in claim 1, wherein the volatile solvent system comprises water.

12. A formulation as in claim 1, wherein the volatile solvent system is substantially free of water.

13. A formulation as in claim 1, wherein the volatile solvent system includes at least member selected from the group consisting of ethanol, isopropyl alcohol, and combinations thereof.

14. A formulation as in claim 1, wherein the solidifying agent is present in the solidified layer at least at 20% by weight after substantially all of the volatile solvent system has evaporated.

15. A formulation as in claim 1, wherein the non-volatile solvent system is present in the solidified layer at least at 20% by weight after substantially all of the volatile solvent system has evaporated.

16. A formulation as in claim 1, wherein the volatile solvent system includes at least one solvent more volatile than water, and includes at least one member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

17. A formulation as in claim 1, wherein the volatile solvent system includes at least one solvent more volatile than water, and includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

18. A formulation as in claim 1, wherein the non-volatile solvent system includes multiple non-volatile solvents admixed together which, along with other ingredients in the formulation, forms a formulation which solidifies onto the skin and delivers the drug at therapeutically effective rates over a sustained period of time.

19. A formulation as in claim 1, wherein the non-volatile solvent system comprises at least one solvent selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

20. A formulation as in claim 1, wherein the non-volatile solvent system includes at least one solvent selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

21. A formulation as in claim 1, wherein the non-volatile solvent system includes at least one solvent selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugarsm ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

22. A formulation as in claim 1, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

23. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, and combinations thereof.

24. A formulation as in claim 1, wherein the solidifying agents includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

25. A formulation as in claim 1, wherein the drug includes at least one member from a class of drugs selected from the group consisting of non-steroidal anti-inflammatory drugs (NSAIDs), COX inhibitors, local anesthetics, 5HT-2A receptor antagonists, and steroids, prodrugs thereof, and combinations thereof.

26. A formulation as in claim 1, wherein the drug includes at least one member selected from the group consisting of ketoprofen, diclofenac, ketanserin, and combinations thereof.

27. A formulation as in claim 1, wherein the drug includes at least one member selected from the group consisting of lidocaine, ropivacaine, bupivacaine, tetracaine, and combinations thereof.

28. A formulation as in claim 1, wherein the drug includes a local anesthetic in base form.

29. A formulation as in claim 1, wherein the drug includes a non-steroidal anti-inflammatory drug and the non-volatile solvent system is capable of generating a flux for the non-steroidal anti-inflammatory drug of at least 1 μg/cm²/hour.

30. A formulation as in claim 1, wherein the drug includes a non-steroidal anti-inflammatory drug and the solidified layer is capable of generating a flux for the non-steroidal anti-inflammatory drug of at least 1 μg/cm²/hour.

31. A formulation as in claim 1, wherein the drug includes a local anesthetic and the non-volatile solvent system is capable of generating a flux for the local anesthetic of at least 5 μg/cm²/hour.

32. A formulation as in claim 1, wherein the drug includes a local anesthetic and the solidified layer is capable of generating a flux for the local anesthetic of at least 5 μg/cm²/hour.

33. A formulation as in claim 1, wherein the solidified layer is sufficiently flexible and adhesive to the skin such that when applied to the skin at a human joint, the solidified layer will remain substantially intact on the skin upon bending of the joint.

34. A formulation as in claim 1, wherein the volatile solvent system comprises a volatile solvent whose boiling point is below 20° C.

35. A formulation as in claim 34, wherein the volatile solvent with the boiling point below 20° C. is completely dissolved in the formulation.

36. A formulation as in claim 34, wherein the volatile solvent with the boiling point below 20° C. is included in the formulation as a propellant for pressurized spray-on application.

37. A formulation as in claim 34, wherein the volatile solvent with the boiling point below 20° C. is a hydrofluorocarbon.

38. The formulation as in claim 34, wherein the at least one solvent whose boiling point is below 20 C is selected from the group consisting of dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof.

39. A formulation as in claim 1, wherein the formulation is formulated to deliver the drug at a therapeutically effective rate for at least about 2 hours following the formation of the solidified layer.

40. A formulation as in claim 1, wherein the formulation is formulated to deliver the drug at a therapeutically effective rate for at least about 4 hours following the formation of the solidified layer.

41. A formulation as in claim 1, wherein the formulation is formulated to deliver the drug at a therapeutically effective rate for at least about 8 hours following the formation of the solidified layer.

42. A formulation as in claim 1, wherein the formulation is formulated to deliver the drug at a therapeutically effective rate for at least about 12 hours following the formation of the solidified layer.

43. A formulation as in claim 1, wherein the solidifying agent is dispersed in the solvent vehicle.

44. A formulation as in claim 1, wherein the solidifying agent is solvated in the solvent vehicle.

45. A formulation as in claim 1, wherein the weight ratio of the non-volatile solvent system to the solidifying agent is from about 0.1:1 to about 10:1.

46. A formulation as in claim 1, wherein the weight ratio of the non-volatile solvent system to the solidifying agent is from about 0.5:1 to about 2:1.

47. A formulation as in claim 1, wherein the non-volatile solvent system is capable of causing human skin irritation and at least one non-volatile solvent of the non-volatile solvent system is capable of reducing the skin irritation.

48. A formulation as in claim 47, wherein the non-volatile solvent capable of reducing skin irritation includes at least one member selected from the group consisting of glycerin, propylene glycol, honey, and combinations thereof.

49. A formulation as in claim 1, wherein the solidified layer is formed within about 15 minutes of application to the skin surface under standard skin and ambient conditions.

50. A formulation as in claim 1, wherein the solidified layer is formed within 4 minutes of the application to the skin surface under standard skin and ambient conditions.

51. A formulation as in claim 1, wherein the formulation has an initial viscosity prior to skin application from about 100 to about 3,000,000 centipoises.

52. A formulation as in claim 1, wherein the formulation has an initial viscosity prior to skin application from about 1,000 to about 1,000,000 centipoises.

53. A formulation as in claim 1, wherein the weight percentage of the volatile solvent system is from about 10 wt % to about 85 wt %.

54. A formulation as in claim 1, wherein the weight percentage of the volatile solvent system is from about 20 wt % to about 50 wt %.

55. A formulation as in claim 1, wherein the non-volatile solvent system includes multiple non-volatile solvents, and at least one of the non-volatile solvents improves the compatibility of the non-volatile solvent system with the solidifying agent.

56. A formulation as in claim 1, wherein the non-volatile solvent includes at least two non-volatile solvents, and wherein one of the at least two non-volatile solvents is included to improve compatibility with the solidifying agent.

57. A formulation as in claim 1, wherein the solidified layer is coherent, flexible, and continuous.

58. A formulation as in claim 1, wherein the solidified layer, upon formation, is a soft, coherent solid that can be peeled from a skin surface as a single piece or as only a few large pieces relative to the application size.

59. A formulation as in claim 1, wherein the solidified layer is formulated to deliver the drug transdermally.

60. A method of dermally delivering a drug for treating pain or inflammation of joints or muscles, comprising:
a) applying a formulation to a skin surface adjacent to a joint or muscle of a subject suffering from pain or inflammation, the formulation comprising:
i) a drug suitable for treating musculoskeletal pain or inflammation;
ii) a solvent vehicle, comprising:
a volatile solvent system including at least one volatile solvent, and
a non-volatile solvent system including at least one non-volatile solvent, and
iii) a solidifying agent,
wherein the formulation has a viscosity suitable for application and adhesion to the skin surface prior to evaporation of the volatile solvent system;
b) solidifying the formulation to form a solidified layer on the skin surface by at least partial evaporation of the volatile solvent system; and
c) dermally delivering the drug from the solidified layer across the skin surface at therapeutically effective rates for treating the pain or inflammation of joints or muscles over a sustained period of time.

61. A method as in claim 60, wherein the non-volatile solvent system is capable of facilitating transdermal delivery of the drug at therapeutically effective rates over a sustained period of time.

62. A method as in claim 60, wherein the formulation is applied onto a skin area over a wrist, ankle, elbow, or knee.

63. A method as in claim 60, wherein the formulation is applied onto a skin area over a finger or toe joint.

64. A method as in claim 60, wherein the formulation is applied onto a skin area over a back.

65. A method as in claim 60, wherein the formulation is applied onto a skin area over a neck.

66. A method as in claim 60, wherein the step of applying includes applying the adhesive peel-forming formulation at a thickness from about 0.01 mm to about 3 mm.

67. A method as in claim 60, wherein the step of applying includes applying the formulation at a thickness from about 0.05 mm to about 1 mm.

68. A method as in claim 60, wherein the non-volatile solvent system acts as a plasticizer for the solidifying agent.

69. A method as in claim 60, wherein the volatile solvent system comprises water.

70. A method as in claim 60, wherein the volatile solvent system includes at least one member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

71. A method as in claim 60, wherein the volatile solvent system includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

72. A method as in claim 60, wherein the non-volatile solvent system includes multiple non-volatile solvents admixed together which, along with other ingredients in the formulation, forms a formulation whose solidified layer on the skin not only delivers the drug at therapeutically effective rates but also has acceptable adhesion to skin and flexibility over a sustained period of time.

73. A method as in claim 60, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

74. A method as in claim 60, wherein the non-volatile solvent system includes at least one member selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

75. A method as in claim 60, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugarsm ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

76. A method as in claim 60, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

77. A method as in claim 60, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof.

78. A method as in claim 60, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

79. A method as in claim 60, wherein the drug includes at least one member from a class of drugs selected from the group consisting of non-steroidal anti-inflammatory drugs (NSAIDs), COX inhibitors, local anesthetics, 5HT-2A receptor antagonists, and steroids, prodrugs thereof, and combinations thereof.

80. A method as in claim 60, wherein the drug includes at least one member selected from the group consisting of ketoprofen, diclofenac, and combinations thereof.

81. A method as in claim 60, wherein the drug includes at least one member selected from the group consisting of lidocaine, ropivacaine, bupivacaine, tetracaine, and combinations thereof.

82. A method as in claim 60, wherein the drug includes multiple pharmaceutically active agents.

83. A method as in claim 60, wherein the solidified layer is sufficiently flexible and adhesive to the skin such that when applied to the skin at a human joint, the solidified layer will remain substantially intact on the skin upon bending of the joint.

84. A method as in claim 60, wherein the formulation is left on the skin for at least about 2 hours following the formation of the solidified layer.

85. A method as in claim 60, wherein the formulation is left on the skin for from 2 to 12 hours following the formation of the solidified layer.

86. A method as in claim 60, wherein the formulation is left on the skin for at least about 12 hours following the formation of the solidified layer.

87. A method as in claim 60, wherein the weight ratio of the non-volatile solvent system to the solidifying agent is from about 0.5:1 to about 2:1.

88. A method as in claim 60, wherein the solidified layer is formed within about 15 minutes of application to the skin surface under standard skin and ambient conditions.

89. A method as in claim 60, wherein the formulation has an initial viscosity prior to skin application from about 100 to about 3,000,000 centipoises.

90. A method as in claim 60, wherein the weight percentage of the volatile solvent system is from about 10 wt % to about 85 wt %.

91. A method as in claim 60, wherein the pain or inflammation is located in a body region including at least one region selected from the group consisting of back, neck, shoulder, and hip.

92. A method as in claim 60, wherein the pain or inflammation is located in a body region including at least one region selected form the group consisting of the finger joints, toes, elbow, knee, or wrist.

93. A method as in claim 60, wherein the solidified layer is coherent, flexible, and continuous.

94. A method as in claim 60, wherein the formulation is sprayed on the skin.

95. A method as in claim 60, wherein the formulation is applied on the skin using a manual pump.

96. A method as in claim 60, wherein the drug is a local anesthetic agent and the solidified layer is capable of generating a flux of the local anesthetic of at least 5 mcg/cm²/h.

97. A method as in claim 60, wherein the drug is a NSAID agent and the non-solidified layer is capable of generating a flux of said NSAID agent of at least 1 mcg/cm²/h.

98. A method as in claim 60, wherein the solidified layer, upon formation, is a soft, coherent solid that is peelable from a skin surface as a single piece or as only a few large pieces relative to the application size.

99. A method as in claim 60, further comprising the step of peeling the solidified layer from the skin after the sustained period of time to remove the solidified layer.

100. A method as in claim 60, further comprising the step of washing the solidified layer form the skin using a solvent after the sustained period of time to remove the solidified layer.

101. A solidified layer for treating musculoskeletal pain or inflammation, comprising:
a) a drug for treating musculoskeletal pain or inflammation,
b) a non-volatile solvent system including at least one non-volatile solvent, and
c) a solidifying agent,
wherein the solidified layer is capable of adhering to a skin surface and delivering the drug across the skin surface at therapeutically effective rates over a sustained period of time.

102. A solidified layer as in claim 101, wherein the solidified layer is formulated to be applied to a skin surface over a wrist, ankle, elbow, or knee.

103. A solidified layer as in claim 101, wherein the solidified layer is formulated to be applied to the skin surface over a finger or toe joint.

104. A solidified layer as in claim 101, wherein the solidified layer is formulated to be applied to the skin surface over a back, neck, shoulder, or hip.

105. A solidified layer as in claim 101, wherein the solidified layer has a thickness from about 0.01 mm to about 3 mm.

106. A solidified layer as in claim 101, wherein the drug includes at least one member from a class of drugs selected from the group consisting of non-steroidal anti-inflammatory drugs (NSAIDs), COX inhibitors, local anesthetics, 5HT-2A receptor antagonists, and steroids, prodrugs thereof, and combinations thereof.

107. A solidified layer as in claim 101, wherein the drug includes at least one member selected from the group consisting of ketoprofen, diclofenac, lidocaine, ropivacaine, bupivacaine, tetracaine, ketanserin, and combinations thereof.

108. A solidified layer as in claim 99, wherein the solidified layer is sufficiently flexible and adhesive to the skin such that when applied to the skin at a human joint, the solidified layer will remain substantially intact on the skin upon bending of the joint.

109. A solidified layer as in claim 101, wherein the solidified layer is formulated to deliver the drug at a therapeutically effective rate for at least about 2 hours.

110. A solidified layer as in claim 101, wherein the formulation is formulated to deliver the drug at a therapeutically effective rate for at least about 12 hours.

111. A solidified layer as in claim 101, wherein the weight ratio of the non-volatile solvent system to the solidifying agent is from about 0.5:1 to about 2:1.

112. A solidified layer as in claim 101, wherein the solidified layer is a soft, coherent solid layer that is peelable from a skin surface as a single piece or as only a few large pieces relative to the application size.

113. A solidified layer as in claim 101, wherein the solidified layer is substantially devoid of water and solvents more volatile than water when the solidified layer contains no more than 10 wt % of water and solvents more volatile than water.

114. A solidified layer as in claim 101, wherein the solidified layer is substantially devoid of water and solvents more volatile than water when the solidified layer contains no more than 5 wt % of water and solvents more volatile than water.

115. A solidified layer as in claim 101, wherein the solidified layer is adhesive to the skin surface on one surface, and is non-adhesive on an opposing surface.

116. A solidified layer as in claim 101, wherein the solidified layer is flux-enabling for the drug.

117. A formulation for treating musculoskeletal pain or inflammation, comprising:
a) ropivacaine;
b) a solvent vehicle, comprising:
i) a volatile solvent system including at least one volatile solvent, and
ii) a non-volatile solvent system including at least one solvent selected from the group consisting of triacetin, span 20, and isostearic acid;
c) a solidifying agent
wherein the ropivacaine is in either base or salt form; wherein the formulation has a viscosity suitable for application to a skin surface prior to evaporation of the volatile solvent system, wherein the formulation applied to the skin surface forms a solidified, coherent, flexible, and continuous layer after at least partial evaporation of the volatile solvent system, and wherein the ropivacaine continues to be delivered at a transdermal flux of at least 5 mcg/cm²/hour after the volatile solvent system is at least substantially all evaporated.

118. A formulation as in claim 117, wherein the ropivacaine continues to be delivered at a transdermal flux of at least 10 mcg/cm²/hour after the volatile solvent system is at least substantially all evaporated.

119. A formulation for treating musculoskeletal pain or inflammation, comprising:
a) lidocaine;
b) a solvent vehicle, comprising:
i) a volatile solvent system including at least one volatile solvent, and
ii) a non-volatile solvent system including at least one solvent selected from the group consisting of propylene glycol and dipropylene glycol; and
c) a solidifying agent,
wherein the lidocaine is in either base or salt form, wherein the formulation has a viscosity suitable for application to a skin surface prior to evaporation of the volatile solvent system, the formulation applied to the skin surface forms a solidified, coherent, flexible and continuous layer after at least partial evaporation of the volatile solvent system, and the lidocaine continues to be delivered at a transdermal flux of at least 20 mcg/cm²/hour after the volatile solvent system is at least substantially all evaporated

120. A formulation for treating musculoskeletal pain or inflammation, comprising:
a) ketoprofen;
b) a solvent vehicle, comprising:
i) a volatile solvent system including at least one volatile solvent, and
ii) a non-volatile solvent system including at least one solvent selected from the group consisting of propylene glycol and glycerol, isostearic acid, triacetin; and
c) a solidifying agent,
wherein the ketoprofen is in either acid or salt form, wherein the formulation has a viscosity suitable for application to a skin surface prior to evaporation of the volatile solvent system, wherein the formulation applied to the skin surface forms a solidified, coherent, flexible and continuous layer after at least partial evaporation of the volatile solvent system, and wherein the ketoprofen continues to be delivered at a transdermal flux of at least 10 mcg/cm²/hour after the volatile solvent system is at least substantially all evaporated.

121. A formulation for treating musculoskeletal pain or inflammation, comprising:
a) tetracaine;
b) a solvent vehicle, comprising:
i) a volatile solvent system including at least one volatile solvent, and
ii) a non-volatile solvent system including at least one solvent selected from the group consisting of propylene glycol and isostearic acid; and
c) a solidifying agent,
wherein the tetracaine is in either base or salt form, wherein the formulation has a viscosity suitable for application to a skin surface prior to evaporation of the volatile solvent system, the formulation applied to the skin surface forms a solidified, coherent, flexible and continuous layer after at least partial evaporation of the volatile solvent system, and the tetracaine continues to be delivered at a transdermal flux of at least 5 mcg/cm²/hour after the volatile solvent system is at least substantially all evaporated.

122. A formulation for treating musculoskeletal pain or inflammation, comprising:
a) lidocaine and tetracaine;
b) a solvent vehicle, comprising:
i) a volatile solvent system including at least one volatile solvent, and
ii) a non-volatile solvent system including at least one solvent selected from the group consisting of propylene glycol, dipropylene glycol, isostearic acid, and combinations thereof; and
c) a solidifying agent,
wherein the tetracaine and lidocaine is in either base or salt form, wherein the formulation has a viscosity suitable for application to a skin surface prior to evaporation of the volatile solvent system, the formulation applied to the skin surface forms a solidified, coherent, flexible and continuous layer after at least partial evaporation of the volatile solvent system, and the tetracaine and lidocaine continue to be delivered at a transdermal flux of at least 5 mcg/cm²/hour, respectively, after the volatile solvent system is at least substantially all evaporated.

123. A formulation for treating musculoskeletal pain or inflammation, comprising:
a) a drug include at least one member from the group consisting of lidocaine, tetracaine, ropivacaine, ketoprofen, diclofenac, and combinations thereof;
b) a solvent vehicle, comprising:
i) a volatile solvent system comprising a volatile solvent whose boiling point is below 20° C., and
ii) a non-volatile solvent system comprising at least one non-volatile solvent; and
c) a solidifying agent,
wherein the formulation has a viscosity suitable for application to a skin surface prior to evaporation of the volatile solvent system, the formulation applied to the skin surface forms a solidified, coherent, flexible and continuous layer after at least partial evaporation of the volatile solvent system, and the drug continues to be delivered at a therapeutically effective rate after the volatile solvent system is at least substantially all evaporated.
